(19) United States
(12) Patent Application Publication
(10) Pub. No.: US 2021/0121675 A1
(43) Pub. Date: Apr. 29, 2021

(54) Systems, Devices, and Methods For Thrombolysis

(71) Applicant: Bard Access Systems, Inc., Salt Lake City, UT (US)

(72) Inventors: Michael Davis, West Jordan, UT (US); Gidon Ofek, Millcreek, UT (US); Austin Russon, South Jordan, UT (US)

(21) Appl. No.: 17/061,283

(22) Filed: Oct. 1, 2020

Related U.S. Application Data
(60) Provisional application No. 62/927,550, filed on Oct. 29, 2019.

(51) Int. Cl. A61M 39/02; A61M 39/22; A61M 39/24; A61M 39/10; A61M 5/178; A61M 5/148

(52) U.S. Cl. CPC A61M 39/0208; A61M 39/223; A61M 39/24; A61M 39/10; A61M 5/178; A61M 5/148; A61M 2039/229; A61M 2202/0427; A61M 2202/0429; A61M 2205/3331; A61M 2205/0233; A61M 2205/058; A61M 2202/045

(57) ABSTRACT

Disclosed herein are systems, devices, and methods for thrombolysis. For example, a medical device for thrombolysis can include a conduit, a supply reservoir, and a waste reservoir. The conduit can be configured to insert into a lumen of a venous access device having an intraluminal clot. The conduit can include a supply lumen configured to convey an aqueous thrombolytic composition from the supply reservoir through an opening in a distal-end portion of the conduit to the intraluminal clot. The waste reservoir can be configured to collect waste from the lumen of the venous access device including fibrin fragments, platelets, red blood cells, or spent solution of the thrombolytic composition used to break down the intraluminal clot. Administering a thrombolytic composition in accordance with the disclosed systems, devices, and methods can break down clots more quickly than at least the common 3-way stopcock method.

### PRIORITY

This application claims the benefit of priority to U.S. Provisional Application No. 62/927,550, filed Oct. 29, 2019, which is incorporated by reference in its entirety into this application.

### BACKGROUND

Venous thrombosis, or formation of thromboses, otherwise known as blood clots, can occur in central veins in which peripherally inserted central catheters (“PICCs”) or other central venous access devices are placed. Intraluminal clots can also occur in the PICCs and other central venous access devices themselves. Such clots are commonly treated with thrombolytic drugs such as alteplase, which is tissue plasminogen activator (“TPA”) produced by recombinant DNA technology. TPA catalyzes conversion of an open form of clot-bound plasminogen into active plasmin, which is a major enzyme responsible for breakdown of fibrin in clots.

In treating a clot to restore patency to a central venous access device or a central vein, a clinician typically forces a solution of TPA into the central venous access device or through the central venous access device and into the central vein proximal of the clot using a 3-way stopcock method. The method requires a drawing step of drawing fluid from the central venous access device or central vein proximal of the clot with a first syringe, thereby creating a partial vacuum, an injecting step of injecting the solution of TPA into the central venous access device or the central vein under the vacuum with a second syringe, and a waiting step of waiting up to at least 20 minutes for the TPA to act on the clot before repeating the drawing and injecting steps. While the 3-way stopcock method can be effective, it can also take over an hour or more to treat a clot due to its size, extent of occlusion, and location in the central access venous device or in the central vein distal thereto.

Disclosed herein are systems, devices, and methods thereof for administering a thrombolytic composition that breaks down clots more quickly than at least the 3-way stopcock method.

### SUMMARY

Disclosed herein is medical device for thrombolysis including, in some embodiments, a conduit and a waste reservoir. The conduit is configured to insert into a lumen of a venous access device. The conduit includes a supply lumen configured to convey an aqueous thrombolytic composition from a supply reservoir thereof through an opening in a distal-end portion of the conduit for administration of the thrombolytic composition to an intraluminal clot in the lumen of the venous access device. A connector coupled to a proximal-end portion of the conduit is configured to fluidly connect the supply reservoir to the supply lumen of the conduit. The waste reservoir is configured to fluidly connect to the lumen of the venous access device. The waste reservoir is configured to collect waste from the lumen of the venous access device including fibrin fragments, platelets, red blood cells, or spent solution of the thrombolytic composition used to break down the intraluminal clot in the lumen of the venous access device.

In some embodiments, the medical device further includes the supply reservoir optionally pre-filled with the thrombolytic composition.

In some embodiments, the supply reservoir is a syringe optionally pre-filled with the thrombolytic composition.

In some embodiments, the supply reservoir is an intravenous (“IV”) bag optionally pre-filled with the thrombolytic composition.

In some embodiments, the waste reservoir is collapsed, partially evacuated, or a combination thereof in an initial state of the waste reservoir for subsequent collection of the waste.

In some embodiments, the waste reservoir includes a one-way valve configured to ensure flow directionality or relieve pressure build-up in an active state of the waste reservoir while the waste reservoir collects the waste.

In some embodiments, the waste reservoir is a waste bag.

In some embodiments, the distal-end portion of the conduit includes an atraumatic tip. The opening in the distal-end portion of the conduit is an opening of the atraumatic tip.

In some embodiments, an abluminal surface of the conduit includes a hydrophilic coating to protect the lumen of the venous access device from incidental damage, improve insertion of the conduit into the venous access device, or improve withdrawal of the conduit into the venous access device.

In some embodiments, the conduit further includes an ultrasonic transducer for application of ultrasound to the intraluminal clot in the lumen of the venous access device to disaggregate fibrin strands of the intraluminal clot.

In some embodiments, the conduit further includes a pressure sensor for detection of back pressure as the conduit and the pressure sensor thereof approaches the intraluminal clot in the lumen of the venous access device.

In some embodiments, the conduit includes conductive leads electrically coupled to one or more electrodes for detection of impedance changes in a space between the conduit and the intraluminal clot in the lumen of the venous access device as the thrombolytic composition breaks down the intraluminal clot.

In some embodiments, the conduit further includes one or more magnetic or electromagnetic elements in the distal-end portion of the conduit for detection of the distal-end portion of the conduit by a tip-location sensor configured to detect the magnetic or electromagnetic elements. The magnetic or electromagnetic elements of the conduit in combination with the tip-location sensor provides a means for ensuring the conduit is not pushed beyond the venous access device unless desired.

In some embodiments, the conduit further includes an auxiliary lumen.

In some embodiments, the auxiliary lumen is configured to accept an ultrasound catheter inserted into the auxiliary lumen. The ultrasound catheter is for application of ultrasound to the intraluminal clot in the lumen of the venous access device. The application of ultrasound disaggregates fibrin strands of the intraluminal clot.

In some embodiments, the auxiliary lumen is configured accept a pressure-sensing catheter inserted into the auxiliary lumen. The pressure-sensing catheter includes a pressure sensor for detection of back pressure as the conduit including the pressure-sensing catheter approaches the intraluminal clot in the lumen of the venous access device.

In some embodiments, the auxiliary lumen is configured accept an impedance catheter inserted into the auxiliary lumen. The impedance catheter includes one or more electrodes for detection of impedance changes in a space between the conduit and the intraluminal clot in the lumen of the venous access device as the thrombolytic composition breaks down the intraluminal clot.

In some embodiments, the auxiliary lumen is configured for application of at least a partial vacuum to a proximal-end portion thereof to induce flow or aspirate through the auxiliary lumen.

In some embodiments, the auxiliary lumen is configured for application of at least a partial vacuum to a proximal-end portion thereof to retrieve the waste or facilitate fluid displacement at the distal-end portion of the conduit inside the lumen of the venous access device.

In some embodiments, the thrombolytic composition includes tissue plasminogen activator (“TPA”) exclusive of any other agents configured to break down the intraluminal clot in the lumen of the venous access device.

In some embodiments, the thrombolytic composition includes one or more agents other than TPA configured to break down the intraluminal clot in the lumen of the venous access device. The one or more agents other than TPA are optionally in combination with TPA.

Also disclosed herein is a medical system for thrombolysis including, in some embodiments, a disposable medical device and one or more reusable medical devices for thrombolysis. The disposable medical device includes a conduit and a waste reservoir. The conduit is configured to insert into a lumen of a venous access device. The conduit includes a supply lumen and an auxiliary lumen in which at least the supply lumen is configured to convey an aqueous thrombolytic composition from a supply reservoir thereof through an opening in a distal-end portion of the conduit for administration of the thrombolytic composition to an intraluminal clot in the lumen of the venous access device. A connector coupled to a proximal-end portion of the conduit is configured to fluidly connect the supply reservoir to the supply lumen of the conduit. The waste reservoir is configured to fluidly connect to the lumen of the venous access device. The waste reservoir is configured to collect waste from the lumen of the venous access device including fibrin fragments, platelets, red blood cells, or spent solution of the thrombolytic composition used to break down the intraluminal clot in the lumen of the venous access device.

In some embodiments, the disposable medical device further includes the supply reservoir optionally pre-filled with the thrombolytic composition.

In some embodiments, the supply reservoir is a syringe optionally pre-filled with the thrombolytic composition.

In some embodiments, the one or more reusable medical devices includes a syringe pump to dispense the thrombolytic composition from the syringe.

In some embodiments, the supply reservoir is an IV bag optionally pre-filled with the thrombolytic composition.

In some embodiments, the one or more reusable medical devices includes a peristaltic pump to pump the thrombolytic composition along infusion-line tubing connected to the IV bag and the conduit.

In some embodiments, the one or more reusable medical devices includes an ultrasound catheter. The auxiliary lumen is configured to accept the ultrasound catheter inserted into the auxiliary lumen. The ultrasound catheter is for application of ultrasound to the intraluminal clot in the lumen of the venous access device. The application of ultrasound disaggregates fibrin strands of the intraluminal clot.

In some embodiments, the one or more reusable medical devices includes a pressure-sensing catheter. The auxiliary lumen is configured to accept the pressure-sensing catheter inserted into the auxiliary lumen. The pressure-sensing catheter is for detection of back pressure with a pressure sensor of the pressure-sensing catheter as the conduit including the pressure-sensing catheter approaches the intraluminal clot in the lumen of the venous access device.

In some embodiments, the one or more reusable medical devices includes an impedance catheter. The auxiliary lumen is configured to accept the impedance catheter inserted into the auxiliary lumen. The impedance catheter is for detection of impedance changes with one or more electrodes of the impedance catheter in a space between the conduit and the intraluminal clot in the lumen of the venous access device as the thrombolytic composition breaks down the intraluminal clot.

Also disclosed herein is a method of a medical device for thrombolysis including, in some embodiments, an inserting step of inserting a conduit of the medical device into a lumen of a venous access device. The conduit includes a supply lumen and, optionally, an auxiliary lumen. The method further includes a first coupling step of coupling together complementary connectors of the venous access device and a waste reservoir of the medical device. The first coupling step fluidly connects the lumen of the venous access device to the waste reservoir. The method further includes a second coupling step of coupling together complementary connectors of the conduit and a supply reservoir of the medical device including aqueous thrombolytic composition. The second coupling step fluidly connects the supply lumen of the conduit to the supply reservoir. The method further includes an administering step of administering the thrombolytic composition to an intraluminal clot in the lumen of the venous access device by conveying the thrombolytic composition through the supply lumen and out of an opening in a distal-end portion of the conduit. The method further includes a collecting step of collecting waste from the lumen of the venous access device in the waste reservoir. The waste includes any combination of fibrin fragments, platelets, red blood cells, or spent solution of the thrombolytic composition used to break down the intraluminal clot in the lumen of the venous access device.

In some embodiments, the method further includes a detecting step of detecting back pressure with a pressure sensor of the conduit as the conduit and the pressure sensor thereof approach the intraluminal clot in the lumen of the venous access device.

In some embodiments, the method further includes an applying step of applying ultrasound to the intraluminal clot in the lumen of the venous access device with an ultrasonic transducer of the conduit to disaggregate fibrin strands of the intraluminal clot for better penetration of the thrombolytic composition.

In some embodiments, the method further includes a detecting step of detecting impedance changes with one or more electrodes of the conduit in a space between the conduit and the intraluminal clot in the lumen of the venous access device as the thrombolytic composition breaks down the intraluminal clot.

In some embodiments, the method further includes an inserting step of inserting a pressure-sensing catheter into the auxiliary lumen of the conduit. The method further includes a detecting step of detecting back pressure with a pressure sensor of the pressure-sensing catheter as the conduit including the pressure-sensing catheter approaches the intraluminal clot in the lumen of the venous access device.

In some embodiments, the method further includes an inserting step of inserting an ultrasound catheter into the auxiliary lumen of the conduit. The method further includes an applying step of applying ultrasound to the intraluminal clot in the lumen of the venous access device to disaggregate fibrin strands of the intraluminal clot for better penetration of the thrombolytic composition.

In some embodiments, the method further includes an inserting step of inserting an impedance catheter into the auxiliary lumen of the conduit. The method further includes a detecting step of detecting impedance changes with one or more electrodes of the impedance catheter in a space between the conduit and the intraluminal clot in the lumen of the venous access device as the thrombolytic composition breaks down the intraluminal clot.

These and other features of the concepts provided herein will become more apparent to those of skill in the art in view of the accompanying drawings and following description, which describe particular embodiments of such concepts in greater detail.

### DRAWINGS

FIG. 1 illustrates a system for thrombolysis in accordance with some embodiments.

FIG. 2 illustrates a conduit of a device for thrombolysis disposed in a lumen of a central venous access device having an intraluminal clot in accordance with some embodiments.

FIG. 3A illustrates a first transverse cross section of the conduit in accordance with some embodiments.

FIG. 3B illustrates a second transverse cross section of the conduit in accordance with some embodiments.

FIG. 3C illustrates a third transverse cross section of the conduit in accordance with some embodiments.

FIG. 3D illustrates a fourth transverse cross section of the conduit in accordance with some embodiments.

FIG. 3E illustrates a fifth transverse cross section of the conduit in accordance with some embodiments.

FIG. 3F illustrates a sixth transverse cross section of the conduit in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

As set forth above, a clinician typically forces a solution of TPA into a central venous access device or through the central venous access device and into a central vein proximal of a clot using a 3-way stopcock method when treating the clot to restore patency to the central venous access device or the central vein. The method requires a drawing step of drawing fluid from the central venous access device or the central vein proximal of the clot with a first syringe, thereby creating a partial vacuum, an injecting step of injecting the solution of TPA into the central venous access device or the central vein under the vacuum with a second syringe, and a waiting step of waiting up to at least 20 minutes for the TPA to act on the clot before repeating the drawing and injecting steps. While the 3-way stopcock method can be effective, it can also take over an hour or more to treat a clot due to its size, extent of occlusion, and location in the central access venous device or in the central vein distal thereto.

Disclosed herein are systems, devices, and methods thereof for administering a thrombolytic composition that breaks down clots more quickly than at least the 3-way stopcock method.

### Systems and Devices

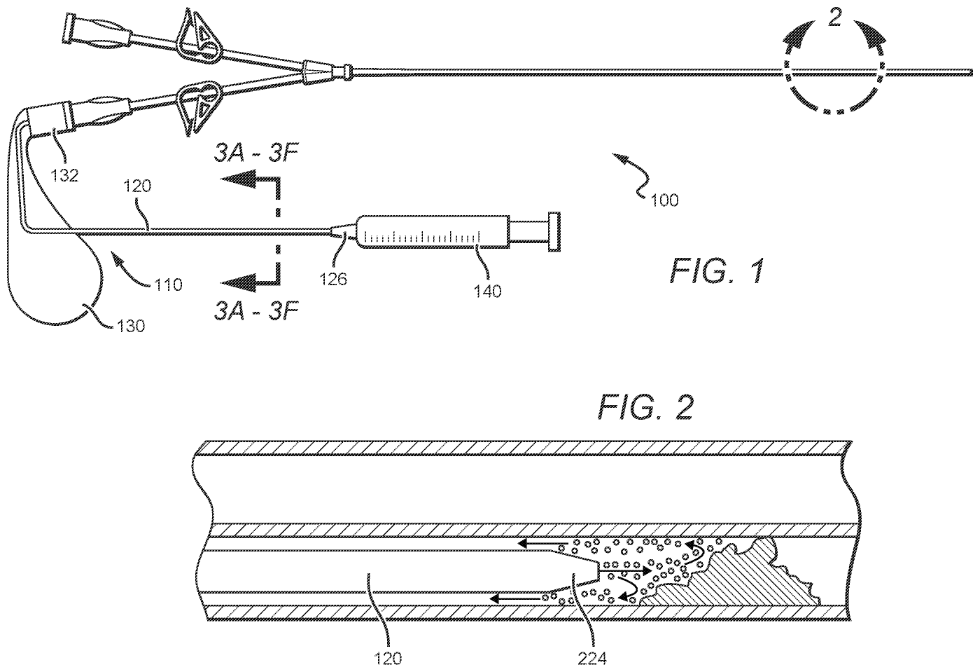

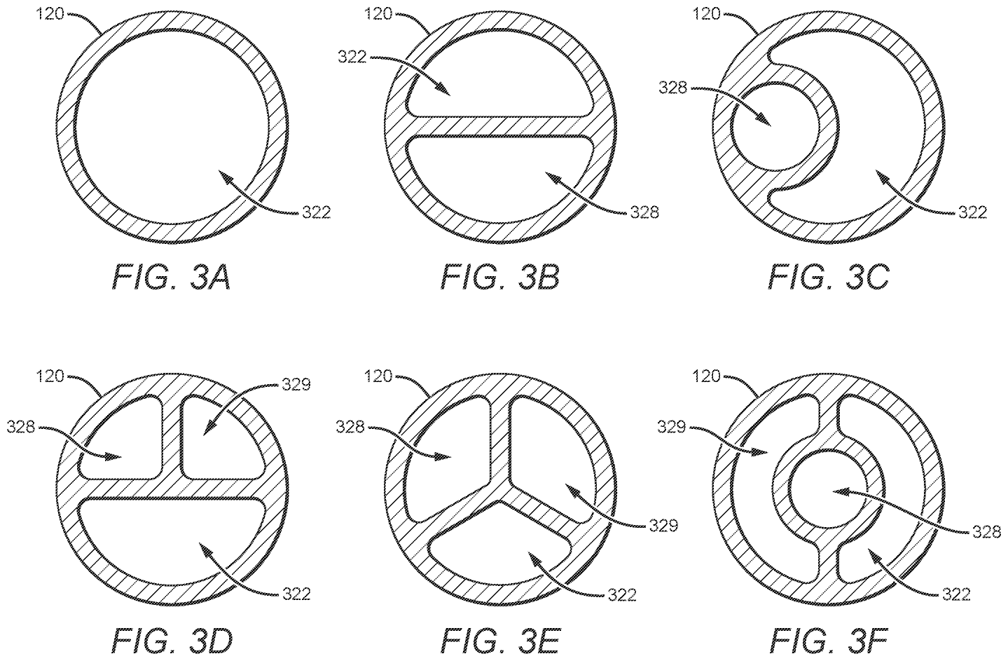

FIG. 1 illustrates a system 100 for thrombolysis in accordance with some embodiments. FIG. 2 illustrates a conduit 120 of a device 110 for thrombolysis disposed in a lumen of a central venous access device having an intraluminal clot in accordance with some embodiments. FIGS. 3A-3F illustrate various transverse cross sections of the conduit 120 in accordance with different embodiments of the conduit 120.

Beginning with the device 110 for thrombolysis, the device 110 includes the conduit 120, a waste reservoir 130, and, optionally, a supply reservoir 140 as shown in FIG. 1. In accordance with description set forth below for the waste reservoir 130 and the supply reservoir 140, neither the waste reservoir 130 nor the supply reservoir 140 is limited to that shown in FIG. 1. In addition, it should be understood the supply reservoir 140 is not optionally used with the device 110 but optionally provided with the device 110 such as in a kit including the device 110.

As shown in FIG. 2, the conduit 120 is configured to insert into a lumen of a venous access device such as a central venous catheter (“CVC”) or a peripherally inserted central catheter (“PICC”). The conduit 120 includes a supply lumen 322 configured to convey an aqueous thrombolytic composition from the supply reservoir 140 through an opening in a distal-end portion of the conduit 120 for administration of the thrombolytic composition to an intraluminal clot in the lumen of the venous access device or a mural thrombus or an occlusive thrombus in a vein such as a central vein of a patient.

The conduit 120 can be made of a metal such as nitinol or a polymeric material such as polyamide. As such, the conduit 120 can be semi-rigid. Such a conduit can include an atraumatic tip 224 in the distal-end portion thereof. Indeed, the opening in the distal-end portion of the conduit 120 is an opening of the atraumatic tip 224 in at least some embodiments. In addition, an abluminal surface of the conduit 120 can include a hydrophilic coating. The atraumatic tip 224 and the hydrophilic coating are configured to protect a lumen of a venous access device or a vein of a patient from incidental damage when the conduit 120 is disposed therein or advanced therethrough. Indeed, the hydrophilic coating can improve insertion of the conduit into the venous access device or improve withdrawal of the conduit from the venous access device.

The conduit 120 can include one or more magnetic or electromagnetic elements in the distal-end portion of the conduit 120 for detection of the distal-end portion of the conduit 120 by a tip-location sensor configured to detect the magnetic or electromagnetic elements. Such a tip-location sensor is set forth in U.S. Pat. No. 8,388,541, the disclosure of which is incorporated herein in its entirety. The magnetic or electromagnetic elements of the conduit 120 in combination with the tip-location sensor provides a means for ensuring the conduit is not pushed beyond the venous access device unless desired.

The conduit 120 can include a connector 126 such as a female Luer connector coupled to a proximal-end portion of the conduit 120. Such a connector is configured to fluidly connect the supply reservoir 140 to the supply lumen 322 of the conduit.

The supply reservoir 140 can include, but is not limited to, a syringe or an IV bag. For example, the supply reservoir 140 shown in FIG. 1 is a syringe. The supply reservoir 140 is optionally pre-filled with the thrombolytic composition such as in the kit set forth above. In other embodiments, the thrombolytic composition in, for example, a container such as a septum-stoppered bottle is either separately provided (e.g., in the kit set forth above) or separately obtained.

The thrombolytic composition can include TPA exclusive of any other agents configured to break down an intraluminal clot in a lumen of the venous access device or a mural thrombus or an occlusive thrombus in a vein of a patient. The thrombolytic composition can include one or more agents other than TPA configured to break down the intraluminal clot in the lumen of the venous access device or the mural thrombus or the occlusive thrombus in the vein of a patient. The one or more agents other than TPA can optionally be in combination with TPA.

The waste reservoir 130 can include, but is not limited to, a waste bag or a rigidly sided waste container configured to collect waste from a lumen of a venous access device or a vein of a patient. For example, the waste reservoir 130 shown in FIG. 1 is a waste bag. Depending upon its construction, the waste reservoir 130 can be collapsed, partially evacuated, or a combination thereof in an initial state of the waste reservoir 130 for subsequent collection of the waste. Alternatively of additionally, the waste reservoir 130 includes a one-way valve configured to ensure flow directionality or relieve pressure build-up in an active state of the waste reservoir 130 while the waste reservoir collects the waste.

The waste reservoir 130 can include a connector 132 such as a female Luer connector coupled to a distal-end portion of the waste reservoir 130 or tubing extending from the distal-end portion of the waste reservoir 130. Such a connector is configured to mate with a complementary connector (e.g., a male Luer connector) on a venous access device to fluidly connect the waste reservoir 130 and a lumen of the venous access device. In addition, the waste reservoir 130, the connector 132, or both the waste reservoir 130 and the connector 132 are configured with the conduit 120 passing therethrough.

Waste from a lumen of a venous access device or a vein of a patient can include fibrin fragments, platelets, red blood cells, or spent solution of the thrombolytic composition used to break down the intraluminal clot in the lumen of the venous access device or the mural thrombus or occlusive thrombus in the vein of the patient.

As shown in FIGS. 3A-3F, the conduit 120 can be a monoluminal conduit or a multi-luminal conduit. The multiluminal embodiment of the conduit 120 includes, for example, a diluminal conduit or a triluminal conduit including at least an auxiliary lumen 328. The auxiliary lumen 328 can be configured for application of at least a partial vacuum to a proximal-end portion thereof to induce flow through the auxiliary lumen 328, aspirate through the auxiliary lumen 328, retrieve the waste through the auxiliary lumen 328, or facilitate fluid displacement at the distal-end portion of the conduit 120 inside the lumen of the venous access device. Alternatively or additionally, the auxiliary lumen 328 is configured as set forth below to accommodate another medical device such as a catheter inserted into the auxiliary lumen 328.

It should be understood that assignment of the lumens of the diluminal and triluminal embodiments of the conduit 120 as the supply lumen 322, the auxiliary lumen 328, or the like is arbitrary. For example, while the smaller lumen and the larger lumen of the lumens of the conduit 120 in FIG. 3C are respectively referenced herein as the supply lumen 322 and the auxiliary lumen 328, the smaller lumen and the larger lumen can alternatively be the auxiliary lumen 328 and the supply lumen 322, respectively.

The monoluminal embodiment of the conduit 120 is intended for administration of the thrombolytic composition to an intraluminal clot in a lumen of a venous access device. The supply lumen 322 of a such a conduit is configured to convey the thrombolytic composition from the supply reservoir 140 through the opening in the distal-end portion of the conduit 120 to the intraluminal clot in the lumen of the venous access device. As shown in FIG. 2, waste including that of fibrin fragments, platelets, red blood cells, or spent solution of the thrombolytic composition used to break down the intraluminal clot in the lumen of the venous access device is conveyed from the intraluminal clot along the lumen of the venous access device to the waste reservoir 130.

The diluminal embodiment of the conduit 120 is intended for administration of the thrombolytic composition to an intraluminal clot in a lumen of a venous access device or a mural thrombus or an occlusive thrombus in a vein of the patient.

With respect to administration of the thrombolytic composition to the intraluminal clot in the lumen of the venous access device, the supply lumen 322 of the diluminal embodiment of the conduit 120 is configured to convey the thrombolytic composition from the supply reservoir 140 through the opening in the distal-end portion of the conduit 120 to the intraluminal clot in the lumen of the venous access device. As set forth in more detail below, the auxiliary lumen 328 is configured to accommodate another medical device such as a catheter inserted into the auxiliary lumen 328. As shown in FIG. 2, waste including that of fibrin fragments, platelets, red blood cells, or spent solution of the thrombolytic composition used to break down the intraluminal clot in the lumen of the venous access device is conveyed from the intraluminal clot along the lumen of the venous access device to the waste reservoir 130.

With respect to administration of the thrombolytic composition to the mural thrombus or the occlusive thrombus in the vein of the patient, the supply lumen 322 of the diluminal embodiment of the conduit 120 is configured to convey the thrombolytic composition from the supply reservoir 140 through the opening in the distal-end portion of the conduit 120 to the mural thrombus or the occlusive thrombus in the vein of the patient. Waste including that of fibrin fragments, platelets, red blood cells, or spent solution of the thrombolytic composition used to break down the mural thrombus or the occlusive thrombus in the vein of the patient is conveyed from the mural thrombus or the occlusive thrombus along the auxiliary lumen 328 of the conduit 120 to the waste reservoir 130.

The triluminal embodiment of the conduit 120 is intended for administration of the thrombolytic composition to a mural thrombus or an occlusive thrombus in a vein of the patient. The supply lumen 322 of a such a conduit is configured to convey the thrombolytic composition from the supply reservoir 140 through the opening in the distal-end portion of the conduit 120 to the mural thrombus or the occlusive thrombus in the vein of the patient. As set forth in more detail below, the auxiliary lumen 328 is configured to accommodate another medical device such as a catheter inserted into the auxiliary lumen 328. Waste including that of fibrin fragments, platelets, red blood cells, or spent solution of the thrombolytic composition used to break down the mural thrombus or the occlusive thrombus in the vein of the patient is conveyed from the mural thrombus or the occlusive thrombus along another auxiliary lumen 329 of the conduit 120 to the waste reservoir 130.

As set forth above, the auxiliary lumen 328 can be configured to accommodate another medical device such as a catheter inserted into the auxiliary lumen 328.

In a first example, the auxiliary lumen 328 can be configured to accept an ultrasound catheter inserted into the auxiliary lumen 328. The ultrasound catheter is for application of ultrasound to an intraluminal clot in a lumen of the venous access device or a mural thrombus or an occlusive thrombus in a vein of a patient. The application of ultrasound reversibly disaggregates fibrin strands of the intraluminal clot, the mural thrombus, or the occlusive thrombus, which exposed more the clot or thrombus to the thrombolytic composition.

Whether or not the conduit 120 includes the auxiliary lumen 328, the conduit 120, itself, can include an ultrasonic transducer for application of ultrasound to the intraluminal clot in the lumen of the venous access device to disaggregate the fibrin strands of the intraluminal clot. Such a conduit obviates a need to use the ultrasound catheter.

In a second example, the auxiliary lumen 328 can be configured to accept a pressure-sensing catheter inserted into the auxiliary lumen 328. The pressure-sensing catheter includes a pressure sensor for detection of back pressure as the conduit 120 including the pressure-sensing catheter approaches an intraluminal clot in a lumen of a venous access device or a mural thrombus or an occlusive thrombus in a vein of a patient.

Whether or not the conduit 120 includes the auxiliary lumen 328, the conduit 120, itself, can include a pressure sensor for detection of back pressure as the conduit and the pressure sensor thereof approaches the intraluminal clot in the lumen of the venous access device. Such a conduit obviates a need to use the pressure-sensing catheter.

In a third example, the auxiliary lumen 328 can be configured to accept an impedance catheter inserted into the auxiliary lumen 328. The impedance catheter includes one or more electrodes for detection of impedance changes in a space between the conduit 120 and an intraluminal clot in a lumen of a venous access device or a mural thrombus or an occlusive thrombus in a vein of a patient as the thrombolytic composition breaks down the intraluminal clot. As an alternative to the impedance catheter, the conduit 120, itself, includes conductive leads (e.g., wires, conductive paint, etc.) electrically coupled to one or more electrodes for detection of impedance changes in the space between the conduit 120 and the intraluminal clot in the lumen of the venous access device or the mural thrombus or the occlusive thrombus in the vein of the patient as the thrombolytic composition breaks down the intraluminal clot.

Continuing with the system 100 for thrombolysis, the system 100 includes a disposable medical device and one or more reusable medical devices for the thrombolysis. The disposable device includes an embodiment of the device 110 set forth above. The one or more reusable medical device are selected from at least a syringe pump to dispense the thrombolytic composition from the syringe when the supply reservoir 140 is the syringe, a peristaltic pump to pump the thrombolytic composition along infusion-line tubing connected to the IV bag when the supply reservoir 140 is the IV bag, an ultrasound catheter, a pressure-sensing catheter, and an impedance catheter.

With respect to the foregoing infusion-line tubing, which is considered part of the disposable medical device, the infusion-line tubing can include one or more ports for combining the thrombolytic composition with one or more other solutions (e.g., saline) or selectively supplying the thrombolytic composition or one or more other solutions (e.g., saline).

### Methods

A method of a medical device for thrombolysis such as the device 110 includes an inserting step of inserting the conduit 120 into a lumen of a venous access device.

The method further includes a first coupling step of coupling together complementary connectors of the venous access device and the waste reservoir 130. The first coupling step fluidly connects the lumen of the venous access device to the waste reservoir 130.

The method further includes a second coupling step of coupling together complementary connectors of the conduit 120 and the supply reservoir 140 including the aqueous thrombolytic composition. The second coupling step fluidly connects the supply lumen 322 of the conduit 120 to the supply reservoir 140.

The method further includes an administering step of administering the thrombolytic composition to an intraluminal clot in the lumen of the venous access device by conveying the thrombolytic composition through the supply lumen 322 and out of the opening in the distal-end portion of the conduit 120.

The method further includes a collecting step of collecting waste from the lumen of the venous access device in the waste reservoir 130. The waste includes any combination of fibrin fragments, platelets, red blood cells, or spent solution of the thrombolytic composition used to break down the intraluminal clot in the lumen of the venous access device.

The method can further include an inserting step of inserting a pressure-sensing catheter into the auxiliary lumen 328 of the conduit 120. The method further includes a detecting step of detecting back pressure with a pressure sensor of the pressure-sensing catheter as the conduit 120 including the pressure-sensing catheter approaches the intraluminal clot in the lumen of the venous access device. As an alternative of the inserting and detecting steps, the method can further include a detecting step of detecting back pressure with the pressure sensor of the conduit 120 as the conduit 120 and the pressure sensor thereof approach the intraluminal clot in the lumen of the venous access device.

The method can further include an inserting step of inserting an ultrasound catheter into the auxiliary lumen 328 of the conduit 120. The method further includes an applying step of applying ultrasound to the intraluminal clot in the lumen of the venous access device to disaggregate fibrin strands of the intraluminal clot for better penetration of the thrombolytic composition. As an alternative of the inserting and applying steps, the method can further include an applying step of applying ultrasound to the intraluminal clot in the lumen of the venous access device with the ultrasonic transducer of the conduit 120 to disaggregate the fibrin strands of the intraluminal clot for better penetration of the thrombolytic composition.

The method further includes an inserting step of inserting an impedance catheter into the auxiliary lumen 328 of the conduit 120. The method further includes a detecting step of detecting impedance changes with one or more electrodes of the impedance catheter in a space between the conduit 120 and the intraluminal clot in the lumen of the venous access device as the thrombolytic composition breaks down the intraluminal clot. As an alternative of the inserting and detecting steps, the method can further include a detecting step of detecting impedance changes with the one or more electrodes of the conduit 120 in the space between the conduit and the intraluminal clot in the lumen of the venous access device as the thrombolytic composition breaks down the intraluminal clot.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A medical device for thrombolysis, comprising:
a conduit configured to insert into a lumen of a venous access device, the conduit including a supply lumen configured to convey an aqueous thrombolytic composition from a supply reservoir thereof through an opening in a distal-end portion of the conduit for administration of the thrombolytic composition to an intraluminal clot in the lumen of the venous access device;
a connector coupled to a proximal-end portion of the conduit, the connector configured to fluidly connect the supply reservoir to the supply lumen of the conduit; and
a waste reservoir configured to fluidly connect to the lumen of the venous access device, the waste reservoir configured to collect waste from the lumen of the venous access device including fibrin fragments, platelets, red blood cells, or spent solution of the thrombolytic composition used to break down the intraluminal clot in the lumen of the venous access device.

2. The medical device of claim 1, further comprising the supply reservoir optionally pre-filled with the thrombolytic composition.

3. The medical device of claim 2, wherein the supply reservoir is a syringe optionally pre-filled with the thrombolytic composition.

4. The medical device of claim 2, wherein the supply reservoir is an intravenous (“IV”) bag optionally pre-filled with the thrombolytic composition.

5. The medical device of claim 1, wherein the waste reservoir is collapsed, partially evacuated, or a combination thereof in an initial state of the waste reservoir for subsequent collection of the waste.

6. The medical device of claim 1, wherein the waste reservoir includes a one-way valve configured to ensure flow directionality or relieve pressure build-up in an active state of the waste reservoir while the waste reservoir collects the waste.

7. The medical device of claim 1, wherein the waste reservoir is a waste bag.

8. The medical device of claim 1, wherein the distal-end portion of the conduit includes an atraumatic tip, the opening in the distal-end portion of the conduit being an opening of the atraumatic tip.

9. The medical device of claim 1, wherein an abluminal surface of the conduit includes a hydrophilic coating to protect the lumen of the venous access device from incidental damage, improve insertion of the conduit into the venous access device, or improve withdrawal of the conduit from the venous access device.

10. The medical device of claim 1, wherein the conduit further includes an ultrasonic transducer for application of ultrasound to the intraluminal clot in the lumen of the venous access device to disaggregate fibrin strands of the intraluminal clot.

11. The medical device of claim 1, wherein the conduit further includes a pressure sensor for detection of back pressure as the conduit and the pressure sensor thereof approaches the intraluminal clot in the lumen of the venous access device.

12. The medical device of claim 1, wherein the conduit includes conductive leads electrically coupled to one or more electrodes for detection of impedance changes in a space between the conduit and the intraluminal clot in the lumen of the venous access device as the thrombolytic composition breaks down the intraluminal clot.

13. The medical device of claim 1, wherein the conduit further includes one or more magnetic or electromagnetic elements in the distal-end portion of the conduit for detection of the distal-end portion of the conduit by a tip-location sensor configured to detect the magnetic or electromagnetic elements, thereby providing a means for ensuring the conduit is not pushed beyond the venous access device unless desired.

14. The medical device of claim 1, wherein the conduit further includes an auxiliary lumen.

15. The medical device of claim 14, wherein the auxiliary lumen is configured to accept an ultrasound catheter inserted into the auxiliary lumen for application of ultrasound to the intraluminal clot in the lumen of the venous access device to disaggregate fibrin strands of the intraluminal clot.

16. The medical device of claim 14, wherein the auxiliary lumen is configured accept a pressure-sensing catheter inserted into the auxiliary lumen, the pressure-sensing catheter including a pressure sensor for detection of back pressure as the conduit including the pressure-sensing catheter approaches the intraluminal clot in the lumen of the venous access device.

17. The medical device of claim 14, wherein the auxiliary lumen is configured accept an impedance catheter inserted into the auxiliary lumen, the impedance catheter including one or more electrodes for detection of impedance changes in a space between the conduit and the intraluminal clot in the lumen of the venous access device as the thrombolytic composition breaks down the intraluminal clot.

18. The medical device of claim 14, wherein the auxiliary lumen is configured for application of at least a partial vacuum to a proximal-end portion thereof to induce flow or aspirate through the auxiliary lumen.

19. The medical device of claim 14, wherein the auxiliary lumen is configured for application of at least a partial vacuum to a proximal-end portion thereof to retrieve the waste or facilitate fluid displacement at the distal-end portion of the conduit inside the lumen of the venous access device.

20. The medical device of claim 1, wherein the thrombolytic composition includes tissue plasminogen activator (“TPA”) exclusive of any other agents configured to break down the intraluminal clot in the lumen of the venous access device.

21. The medical device of claim 1, wherein the thrombolytic composition includes one or more agents other than tissue plasminogen activator (“TPA”) configured to break down the intraluminal clot in the lumen of the venous access device, the one or more agents optionally in combination with TPA.

22. A medical system for thrombolysis, comprising:
a disposable medical device for the thrombolysis including:
a conduit configured to insert into a lumen of a venous access device, the conduit including a supply lumen and an auxiliary lumen in which at least the supply lumen is configured to convey an aqueous thrombolytic composition from a supply reservoir thereof through an opening in a distal-end portion of the conduit for administration of the thrombolytic composition to an intraluminal clot in the lumen of the venous access device;
a connector coupled to a proximal-end portion of the conduit, the connector configured to fluidly connect the supply reservoir to the supply lumen of the conduit; and
a waste reservoir configured to fluidly connect to the lumen of the venous access device, the waste reservoir configured to collect waste from the lumen of the venous access device including fibrin fragments, platelets, red blood cells, or spent solution of the thrombolytic composition used to break down the intraluminal clot in the lumen of the venous access device; and
one or more reusable medical devices for the thrombolysis.

23. The medical system of claim 22, wherein the disposable medical device further comprises the supply reservoir optionally pre-filled with the thrombolytic composition.

24. The medical system of claim 23, wherein the supply reservoir is a syringe optionally pre-filled with the thrombolytic composition.

25. The medical system of claim 24, wherein the one or more reusable medical devices comprises a syringe pump to dispense the thrombolytic composition from the syringe.

26. The medical system of claim 23, wherein the supply reservoir is an intravenous (“IV”) bag optionally pre-filled with the thrombolytic composition.

27. The medical system of claim 26, wherein the one or more reusable medical devices comprises a peristaltic pump to pump the thrombolytic composition along infusion-line tubing connected to the IV bag and the conduit.

28. The medical system of claim 22, wherein the one or more reusable medical devices includes an ultrasound catheter, the auxiliary lumen configured to accept the ultrasound catheter inserted into the auxiliary lumen for application of ultrasound to the intraluminal clot in the lumen of the venous access device to disaggregate fibrin strands of the intraluminal clot.

29. The medical system of claim 22, wherein the one or more reusable medical devices includes a pressure-sensing catheter, the auxiliary lumen configured to accept the pressure-sensing catheter inserted into the auxiliary lumen for detection of back pressure with a pressure sensor of the pressure-sensing catheter as the conduit including the pressure-sensing catheter approaches the intraluminal clot in the lumen of the venous access device.

30. The medical system of claim 22, wherein the one or more reusable medical devices includes an impedance catheter, the auxiliary lumen configured to accept the impedance catheter inserted into the auxiliary lumen for detection of impedance changes with one or more electrodes of the impedance catheter in a space between the conduit and the intraluminal clot in the lumen of the venous access device as the thrombolytic composition breaks down the intraluminal clot.

31-37. (canceled)
